(19) United States
(12) Patent Application Publication
(10) Pub. No.: US 2013/0324502 A1
(43) Pub. Date: Dec. 5, 2013

(54) NOVEL FORMULATIONS FOR DERMAL, TRANSDERMAL AND MUCOSAL USE 1

(71) Applicants: Åke Lindahl, Malmo (SE); Peter Kaufmann, Jarna (SE)

(72) Inventors: Åke Lindahl, Malmo (SE); Peter Kaufmann, Jarna (SE)

(73) Assignee: MOBERG PHARMA AB, Bromma (SE)

(21) Appl. No.: 13/984,684

(22) PCT Filed: Feb. 10, 2012
(86) PCT No.: PCT/EP2012/052325

§ 371 Date: Aug. 9, 2013

Related U.S. Application Data
(60) Provisional application No. 61/441,346, filed on Feb. 10, 2011.

(51) Int. Cl. A61K 47/14; A61K 47/02; A61K 31/60; A61K 31/235; A61K 31/58; A61K 31/4015; A61K 47/10; A61K 31/395

(52) U.S. Cl. CPC A61K 47/14; A61K 47/10; A61K 47/02; A61K 31/395; A61K 31/235; A61K 31/58; A61K 31/4015; A61K 31/60
USPC 514/163; 514/263.38; 514/714; 514/172; 514/173; 514/424

(57) ABSTRACT

A dermal, transdermal and/or mucosal formulation for topical application on skin, comprising an active pharmaceutical ingredient and a pharmaceutically acceptable solvent, and an anti-solvent; wherein the active pharmaceutical ingredient is soluble in the solvent in the absence of the anti-solvent, and wherein the active pharmaceutical ingredient is substantially in the solid state in the presence of the anti-solvent. A method for increasing the stability of an active pharmaceutical ingredient.

### TECHNICAL FIELD

This description, claims and examples generally relate to novel formulations for dermal, transdermal and/or mucosal application, offering improved stability during storage with maintained, good penetration after application.

### BACKGROUND

There are several different routes for delivering an active pharmaceutical ingredient (API) to a patient in need thereof. These can be divided into oral (including gastric, enteric, and colonic), mucosal (buccal, sublingual, nasal, ophthalmic, vaginal, urethral and anal), pulmonary, transdermal, and injectable (including intravenous, subcutaneous, intramuscular, intraepidural, intracranial, also including implants, inserts, ports and pumps for delivery of an API).

When choosing between different routes of administration, one needs to consider inter alia the properties of the API, its metabolization, intended dosage and dose frequency, and if the drug is to be taken by the patient themselves or administered by a nurse or physician. The present description, claims and examples relates mainly to dermal, transdermal and/or mucosal formulations, which in turn can be subdivided into topical and systemic, depending on whether the API exerts its effect locally, at the site of application, or systemically.

Notably, this description, claims and examples also relate to dermal, transdermal and/or mucosal formulations which may have either a local, i.e. topical effect, or a systemic effect. Dermal, transdermal and/or mucosal formulations in general offer many advantages, such as the possibility of easy and safe self-administration, the avoidance or delay of first-pass metabolism, and the possibility of local, targeted administration, in particular in dermatological indications.

Dermal, as well as transdermal formulations include dermatological formulations, i.e. formulations intended for the alleviation, treatment or prevention of diseases of the skin. For the purpose of this description, also cosmetic formulations are included herein, and defined as formulations intended to alleviate, treat or prevent conditions of the skin, which conditions may, or may not, be considered diseases depending on their severity.

Most dermatological formulations are designed to deliver as much as possible of the API to, into and through the skin. There are mainly two routes for a drug to penetrate the skin, either via the intercellular route, i.e. between the cells, or via the transcellular route, i.e. through the cells. It is generally held that the transcellular route is the main pathway for polar substances. The skin is however an effective barrier, developed during evolution to regulate the inward and outward passage of water and electrolytes, and to protect the body from toxic substances. Most of the barrier function is provided by the stratum corneum, the top layer of the epidermis which mainly consists of flat, dead skin cells.

Achieving good penetration is a challenging task, considering that the stratum corneum effectively limits the rate of penetration. Dermatological formulations must therefore be designed in such a way that maximum penetration is achieved. It is generally understood that a drug needs to be presented in the dissolved state, chemically active, in order to maximize penetration.

For mucosal formulations, achieving good penetration is easier, but the mechanisms of intercellular and transcellular transport apply also here.

In addition to penetration, also stability needs to be considered. The stability of a drug, either of the API itself, or the entire formulation, is influenced by many factors. Notably, many APIs are not chemically stable in solution, which makes formulations where the drug is in solution unsuitable. Non-limiting examples of reactions that will compromise the function of the drug include hydrolysis, esterification, dimerization, conjugation, reduction and oxidation. These stability problems explain, at least in part, the popularity of dry, oral formations, such as compressed tablets, capsules and the like.

The stability of the API in a dermal, dermatological and/or mucosal formulation is of great importance. Stability problems involve the loss of efficacy, the accumulation of potentially toxic degradation products, unacceptable changes of the product appearance, such as color changes, stratification, turbidity etc. Almost regardless of the nature of the stability problem, two main approaches remain. The first, restricting the shelf life of the product, has economical and practical consequences, influencing the price and popularity of the product. In cases where the shelf-life is very short, products need to be discarded, either by the distributor, retailer, or the end-user. The second approach, to tailor the formulation e.g. by adding stabilizers and other excipients, requires considerable skill in order not to compromise other properties of the API.

Ultimately, if the API is not stable, an otherwise efficient and clinically relevant API can perhaps not be used at all. Thus, there is a need for formulations where the API has an improved stability without compromising other properties, such as the penetration.

U.S. Pat. No. 5,145,685 (Dow Corning; Walter J. Carmody) describes a method of treating skin disorders, such as acne, by topically applying to the infected area a mixture of an antimicrobial agent and a volatile low viscosity organosilicon compound. The mixture is entrapped within and dispersed uniformly throughout discrete particles of a hydrophobic macroporous highly cross linked polymer. This patent does not describe dissolution of the active pharmaceutical ingredient upon evaporation of a solvent.

U.S. Pat. No. 5,958,379 (Mika Pharma; Juergen Regenold, Carl Artmann) discloses a dermal, transdermal and/or mucosal formulation containing an easily vaporizable organic solvent, which formulation can be sprayed on the body and which comprises an active substance. The concentration of the active substance increases upon application on the body when the organic solvent vaporizes.

Several patent applications teach the possibility of increasing penetration of an active pharmaceutical ingredient through skin by evaporation of solvents. For example WO 2007070695 (Zars Inc, Zhang Jie et al.) concerns adhesive solidifying formulations, methods of drug delivery, and solidified layers for dermal delivery of a drug. The formulation can include a drug, a solvent vehicle, and a solidifying agent. The solvent vehicle can include a volatile solvent system comprising at least one volatile solvent, and a non-volatile solvent system comprising at least one non-volatile solvent, wherein at least one non-volatile solvent is a flux-enabling non-volatile solvent(s) capable of facilitating the delivery of the drug at therapeutically effective rates over a sustained period of time. The formulation can have a viscosity suitable for application to a skin surface prior to evaporation of the volatile solvents system. When applied to the skin, the formulation can form a solidified layer after at least a portion of the volatile solvent system is evaporated.

WO 2009017767 (Zars Pharma, Sanjay Sharma et al.) discloses adhesive solidifying formulations containing a drug, e.g. minoxidil, a solvent vehicle, and a solidifying agent as disclosed in the above WO 2007070695.

WO2007070679 (Zars Inc., Zhang Jie et al.) concerns solidifying formulations for dermal delivery of a drug for treating pain, such as musculoskeletal pain, inflammation, joint pain, or neuropathic pain. The formulation can include a drug selected from certain drug classes, a solvent vehicle, and a solidifying agent. The solvent vehicle can include a volatile solvent system comprising at least one volatile solvent, and a non-volatile solvent system comprising at least one non-volatile solvent, wherein the evaporation of at least some of the volatile solvent converts the formulation on the skin into a solidified layer and the non-volatile solvent system is capable of facilitating the topical delivery of the drug(s) at therapeutically effective rates over a sustained period of time.

Similarly, also WO2007070643 (Zars Inc., Zhang Jie et al.) concerns solidifying adhesive formulations, methods of drug delivery, and solidified layers for dermal delivery of a drug, focusing on the treatment of various dermatological conditions.

### DEFINITIONS

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise. Also, the term “about” is used to indicate a deviation of at least +/−2% of the given value, preferably +/−5%, and most preferably +/−10% of the numeric values, where applicable. Percentages are given as weight/weight, unless otherwise indicated.

In addition to the above, the following terms will be used:

The term “soluble” refers the ability of the solvent to dissolve an amount of the active pharmaceutical ingredient that is relevant for its pharmacological effect. To illustrate this definition, which is clear to a person skilled in the art, one can look at acetylsalicylic acid (ASA), where frequently a dose of 500 mg is accommodated into one tablet or capsule. In order to replace a solid formulation with a liquid or semi-liquid formulation of about 5 to 10 ml, the solubility of ASA in the solvent used needs to be at least in the range of about 50 to 100 mg/ml. Similarly, a steroid drug such as fludrocortisone is supplied in tablets containing 0.1 mg of the active ingredient. Here, in order to replace a solid formulation of fludrocortisone with a liquid or semi-liquid formulation of about 5 to 10 ml, it will be sufficient if a solubility of fludrocortisone in the range of about 0.01 to about to 0.02 mg/ml is achieved in the solvent.

The terms “stable” and “stability” are used here in relation to the shelf-life of a pharmaceutical product, and are related to the physical change, degradation or chemical decomposition of active pharmaceutical ingredients, which limits the shelf-life of a product. Each active pharmaceutical ingredient has its intrinsic stability, its degradation pathways and degradation products, in part depending on the formulation of which it is part, and the storage conditions. The major mechanisms of chemical degradation include oxidation, hydrolysis/dehydration, isomerization/epimerization, decarboxylation, dimerization/polymerization, photolysis and rearrangements. If a product is termed to be “stable” it means in this context that it can be stored for a prescribed time without any of these mechanisms advancing to the extent that compromises product efficacy and safety.

The skilled person is well familiar with the problems of stability, and recognizes the relative nature of this term, as well as the significant advantages of increasing the stability of a product.

The expression “substantially in solid state in the formulation” defines that the active pharmaceutical ingredient is present in solid state to a major part, or to an extent which significantly increases its stability during storage. Preferably at least 50% of the API in the formulation is in the solid state during storage at the prescribed storage temperature of the particular formulation in question. In practice, storage temperatures in the interval of 2-25° C. are used for pharmaceutical preparations, wherein the lower part of the interval is prescribed for sensitive APIs and/or formulations with stability problems.

In the present formulations, the API is preferably in the solid state at a temperature in the above interval, depending on the type of product and API in question, to an extent of at least 50%, preferably at least 60%, more preferably at least 70%, more preferably at least 80%, more preferably at least 90%.

The terms “solvent” and “solvent system” define one component of the formulation, the liquid or semi-solid phase that contains the API. It is included in this definition, that the “solvent” and/or “solvent system” needs to be compatible with the API as well as with the anti-solvent.

The term “anti-solvent” is used to define a substance, which when present in the formulation, forces the API into the solid state.

Similarly, it is included in this definition that the “anti-solvent” needs to be compatible with the API and with the solvent and/or solvent system in that sense that no unwanted interactions, degradation or chemical reactions occur between the components.

The term “skin” is used in its common, physiological meaning to denote the largest organ of the mammal body, and it is here intended to include the skin of a human or mammal body, including the lips, palms, soles, and lips; whereas the hair and nails are excluded. In veterinary applications, the skin includes both furry and non-furry parts of the animal body, whereas hoofs, cloves and claws are excluded.

The term “mucous membrane” refers to the lining of cavities inside the body, and includes the oral cavity, nasal cavity, larynx, eyelids, vagina, urethra, anus and rectum. While the mucous membranes are continuous with the skin, there are defined borders, such as the vermilion line of the lips. However, for the purpose of this description, only a rough distinction is made between mucous membranes and the skin.

Further, the permeability of the API has been tested in order to investigate the availability of the drug, using standardized laboratory methods described in closer detail in the experimental section.

Finally, by “dermal, dermatological and/or mucosal formulations”, it is intended to include common formulations such as a cream, ointment, paste, lotion, gel, foam and spray.

As used herein, unless stated otherwise, the amounts of components in percent refer to percent by weight and are based on the total weight of the formulation.

### SUMMARY

The problems outlined above, and other problems evident to a skilled person upon study of the present description, claims and examples, are solved by formulations described herein, where the API has an increased stability and which, upon application to the skin or a mucous membrane, provide sufficient penetration of the API.

A general embodiment of the invention is thus a dermal, transdermal, and/or mucosal formulation comprising at least one active pharmaceutical ingredient; a pharmaceutically acceptable solvent and/or solvent system; and a pharmaceutically acceptable anti-solvent, wherein the active pharmaceutical ingredient is substantially in the solid state in said formulation in the presence of said anti-solvent; the active pharmaceutical ingredient is soluble in the solvent and/or solvent system in the absence of said anti-solvent, and the solvent and/or solvent system includes an alcohol.

In the above, the pharmaceutically acceptable solvent and/or solvent system preferably includes an alcohol chosen from mono-, di- and trihydric alcohols, unsaturated aliphatic alcohols and alicyclic alcohols, and combinations thereof.

According to an embodiment, the pharmaceutically acceptable solvent and/or solvent system includes a mono-, di- or trihydric alcohol.

According to another embodiment, the pharmaceutically acceptable solvent and/or solvent system includes an unsaturated aliphatic alcohol.

According to yet another embodiment, the pharmaceutically acceptable solvent and/or solvent system includes an unsaturated alicyclic alcohol.

Preferably, the pharmaceutically acceptable solvent and/or solvent system includes an alcohol chosen from isopropyl alcohol, 1,2-propanediol or propylene glycol, butanediol, pentanediol, hexanediol, polyethylene glycol, oleyl alcohol, cetyl alcohol, and mixtures thereof.

According to another embodiment, freely combinable with any one of the above embodiments, the formulation further comprises a solvent chosen from inositol, xylitol, sorbitol and mannitol.

Further, the pharmaceutically acceptable solvent and/or solvent system is preferably liquid or semi-solid at 25° C. Preferably the pharmaceutically acceptable solvent and/or solvent system has a vapor pressure of less than 1 kPa at 25° C. More preferably the pharmaceutically acceptable solvent and/or solvent system has a vapor pressure of less than 0.8 kPa at 25° C., and most preferably the pharmaceutically acceptable solvent and/or solvent system has a vapor pressure of less than 0.5 kPa at 25° C.

According to an embodiment, the anti-solvent is chosen from pharmaceutically acceptable compounds having vapor pressure of more than 1 kPa at 25° C.

Preferably the anti-solvent comprises at least one compound selected from the group consisting of 3-methyl-2-butanone, acetone, butyl acetate, ethyl acetate, heptanes, methyl ethyl ketone, methyl isobutyl ketone, pentane, propyl acetate, methyl acetate, water, and combinations thereof.

The anti-solvent preferably comprises at least one compound selected from the group consisting of butyl acetate, ethyl acetate, water and combinations thereof. Most preferably, the anti-solvent is an ester. Preferably said ester is selected from the group consisting of methyl acetate, ethyl acetate, butyl acetate and propyl acetate.

According to another embodiment, freely combinable with any one of the above embodiments, the formulation is in the form of a cream, ointment, paste, lotion, gel, foam or spray.

Another embodiment is a method for increasing the stability of an active pharmaceutical ingredient in a dermal and/or mucosal formulation, wherein the active pharmaceutical ingredient is dissolved in a solvent and/or solvent system, whereupon an anti-solvent is added, said anti-solvent being effective to substantially precipitate said active pharmaceutical ingredient, and wherein said solvent and/or solvent system comprises an alcohol.

In the method, the pharmaceutically acceptable solvent and/or solvent system includes an alcohol chosen from mono-, di- and trihydric alcohols, unsaturated aliphatic alcohols and alicyclic alcohols, and combinations thereof.

Preferably, the pharmaceutically acceptable solvent and/or solvent system includes a mono-, di- or trihydric alcohol, an unsaturated aliphatic alcohol, or an unsaturated alicyclic alcohol.

Most preferably, the pharmaceutically acceptable solvent and/or solvent system includes an alcohol chosen from isopropylalcohol, 1,2-propanediol or propyleneglycol, butanediol, pentanediol, hexanediol, polyethylene glycol, oleyl alcohol, cetyl alcohol, and mixtures thereof.

According to another embodiment, freely combinable with any one of the above embodiments, the method further comprises the addition of a solvent chosen from inositol, xylitol, sorbitol and mannitol.

Further, in the method, the pharmaceutically acceptable solvent and/or solvent system is preferably liquid or semi-solid at 25° C. Preferably the pharmaceutically acceptable solvent and/or solvent system has a vapor pressure of less than 1 kPa at 25° C. More preferably the pharmaceutically acceptable solvent and/or solvent system has a vapor pressure of less than 0.8 kPa at 25° C., and most preferably the pharmaceutically acceptable solvent and/or solvent system has a vapor pressure of less than 0.5 kPa at 25° C.

Further according to an embodiment of the method, the anti-solvent is chosen from pharmaceutically acceptable compounds having vapor pressure of more than 1 kPa at 25° C.

Preferably said anti-solvent comprises at least one compound selected from the group consisting of pentane, heptane, ethyl acetate, butyl acetate, propyl acetate, methyl acetate, acetone, methyl ethyl ketone, methyl isobutyl ketone, 3-methyl-2-butanone, and water, or combinations thereof.

Preferably the anti-solvent comprises at least one compound selected from the group consisting of butyl acetate, ethyl acetate, water and combinations thereof. Most preferably the anti-solvent is an ester. Preferably said ester is selected from the group consisting of methyl acetate, ethyl acetate, butyl acetate and propyl acetate.

According to another embodiment of the method, freely combinable with any one of the above embodiments, the formulation is in the form of a cream, ointment, paste, lotion, gel, foam or spray.

One important advantage of the present invention is that the dermal, transdermal and/or mucosal formulation will have a high stability and therefore a long shelf-life.

Another advantage is that, because the active pharmaceutical ingredient is more stable, the pharmaceutical formulation will contain no or significantly less degradation products and impurities when it is used by the consumer. Such degradation products can be harmful to the user, elicit side-effects or irritation, or interact and negatively influence other components of the formulation.

Yet another advantage is that a dermal, transdermal and/or mucosal formulation according to the invention will be cost-efficient. Because the active pharmaceutical ingredient remains active to a greater extent, a lower concentration of the active pharmaceutical ingredient can be used in the dermal, transdermal and/or mucosal formulation. Also the longer shelf-life adds to cost-efficiency, as larger batches can be produced, distributed and stored.

### SHORT DESCRIPTION OF THE FIGURES

Different embodiments of the invention will be described in the description, examples, and claims, and supported by the attached drawings, in which:

FIG. 1 shows the cumulative penetration of salicylic acid across a hoof material membrane (in a Franz cell experimental set-up) for the formulation produced in Example 7; and

FIG. 2 shows the cumulative penetration of piracetam across a full thickness pig skin membrane (according to the Bronaugh procedure) for a commercial piracetam formulation, compared to a piracetam formulation according to an embodiment of the invention.

### DESCRIPTION

In the following, a detailed description of different embodiments of the invention will be provided.

The dermal, transdermal and/or mucosal formulation comprises a suspension of the drug consisting of at least three functional parts; an active pharmaceutical ingredient, a solvent and/or solvent system and a anti-solvent that precipitates the active pharmaceutical ingredient. The solvent and/or solvent system has a lower vapor pressure than the anti-solvent such that the formulation is a suspension when the evaporating component, i.e. the anti-solvent, is present while the formulation turns into a solution when the anti-solvent has evaporated.

The formulations according to different embodiments are intended for local use, in particular for application on skin and mucous membranes or in wounds or in open body cavities for the purpose to treat the skin, mucous membrane, underlying tissue, or for providing a systemic effect. Preferably, the formulation is a dermal, transdermal and/or mucosal formulation for application on the skin, specifically excluding nail tissue.

### The Solvent System

The dermal, transdermal and/or mucosal formulation comprises a solvent and/or solvent system for the active pharmaceutical ingredient. The solvent, and/or solvents in the case of a solvent system, is/are chosen from pharmaceutically acceptable solvents. The solvent preferably has a low evaporation at ambient temperatures as well as normal skin temperatures. This corresponds to a temperature range of about 15 to about 40° C.

Preferably the solvent and/or solvent system has a vapor pressure of less than 1 kPa at 25° C. More preferably the solvent has a vapor pressure of less than 0.8 kPa at 25° C., even more preferably less than 0.5 kPa at 25° C.

According to an embodiment of the invention, said solvent and/or solvent system is an alcohol, preferably a mono-, di- or trihydric alcohol, a polyhydric alcohol, an unsaturated aliphatic alcohol, an unsaturated alicyclic alcohol. In this context, the term “alcohol” is also intended to include other compounds rich in functional hydroxyl groups, such as polyethylene glycol and saccharides.

Detailed examples of monohydric alcohols for use as solvent, and/or for inclusion in a solvent system include, but are not limited to ethanol, isopropanol, 1-propanol, and cetyl alcohol.

Similarly, examples of polyhydric alcohols for use as solvent, and/or for inclusion in a solvent system include, but are not limited to, 3-carbon alcohols such as propylene glycol, and glycerol and mixtures thereof; 4-carbon alcohols such as ethylene glycol, butylene glycol, glycerol, erythritol, threitol and mixtures thereof; 5-carbon alcohols, such as arabitol, xylitol, and ribitol, and mixtures thereof; 6-carbon alcohols, such as dipropylene glycol, hexylene glycol, mannitol, sorbitol, dulcitol, fucitol, and iditol, and mixtures thereof; as well as 12-carbon alcohols, such as isomalt, maltitol, lacitol, and polyglycitol; and mixtures thereof.

Preferred compounds for use as solvent, and/or for inclusion in a solvent system are ethanol, isopropyl alcohol, 1,2-propanediol, butanediol, pentanediol, hexanediol, polyethylene glycol, oleyl alcohol, cetyl alcohol, and mixtures thereof.

Further, a compound as defined above, or mixture thereof, may have a dual function, and may thus also be included in the formulation as part of the solvent system or as an excipient.

### The Active Pharmaceutical Ingredient

The active pharmaceutical ingredient and/or ingredients are selected from a wide range of different compounds, which are soluble in the solvent system, compatible with the solvent and/or solvent system defined above, and suitable for delivery to the skin or a mucosal membrane. This may vary between APIs depending on the physical as well as pharmacological properties of the API, such as the particle size, chemical derivatization (e.g. salt or ester form), but is well understood and predictable by a person skilled in the art.

The amount of active pharmaceutical ingredient present in the invented formulation is determined based on its therapeutic activity and required dose, and is normally in the range of from 0.001% to 10%.

The amount of active pharmaceutical ingredient included in the formulation is chosen depending on the amount of compound needed to achieve its pharmacological activity and the penetrative properties of the drug.

Thus, an active pharmaceutical ingredient which is soluble in the solvent system, compatible with said solvent system, and suitable for delivery to the skin or a mucosal membrane, is chosen from the group consisting of: sympathomimetics, sympatholytics, parasympathomimetics, parasympatholytics, ganglioplegics, myorelaxants, antihypertensives, diuretics, cardiotonics, anti-arythmics, anti-angina drugs, cerebral and peripheric vasodilatators, anti-migraine drugs, anti-histaminic drugs, anti-asthma drugs, thrombolytics, general anaesthetics, anxiolytics, antidepressants, neuroleptics, anti-convulsive drugs, hypothalamo-hypophysis regulators, hypo and hyperthyroidics, corticosteroids, glycemia regulators, hypolipidemia drugs, phosphocalcic metabolism regulators, antipyretics, anti-inflammatory drugs, laxatives, anti-anemia drugs, cutaneous disease drugs, antiparasitic drugs, antibiotics, penicillins, cephalosporins, aminosids, sulfamides, diaminopyrimidines, tetracyclins, macrolides, vancomycin, teicoplanin, rifampicin, fusidic acid, lincosamides, quinolones, anticancer drugs, antiviral drugs, and antifungal drugs.

Similarly, an active pharmaceutical ingredient which is soluble in the solvent system, compatible with said solvent system, and suitable for delivery to the skin or a mucosal membrane, is chosen from the group consisting of: anti-acne agents, anti-gout drugs, local anesthetics, general anesthetics, muscle relaxant drugs, hydrochlorothiazides, angiotensin converting enzyme inhibitors, calcium-channel blockers, anti-angina drugs, anti-migraine drugs, antiemetic drugs, anti-histaminic and anti-asthma drugs, thrombolytics and derivatives thereof, analgesics, salicylic acid and derivatives thereof, nonsteroidal anti-inflammatory agents, antitussive, tricyclic antidepressants, tetracyclic antidepressants, antidepressants, monoamine oxidase inhibitors, serotonin precursors, lithium salts, and tranquilizers.

Furthermore, an active pharmaceutical ingredient which is soluble in the solvent system, compatible with said solvent system, and suitable for delivery to the skin or a mucosal membrane, is chosen from the group consisting of: anorectics, nootropics, hypnotics, analeptics, tricyclic neuroleptics, neuroleptics, benzamide neuroleptics, anti-psychotic, anti-convulsive drugs, hypothalamo-hypophysis regulators, anti hypo- and anti hyperthyroidy drugs, glycemia regulators, hypolipidemia drugs, phosphocalcic metabolism regulators, anti-inflammatory drugs, antisecretive gastric drugs, anti-anemia drugs, cutaneous disease drugs; alpha antagonist drugs, antiparasitic drugs, antineoplasic drugs, antiviral drugs, and antifungal drugs.

Further, an active pharmaceutical ingredient which is soluble in the solvent system, compatible with said solvent system, and suitable for delivery to the skin or a mucosal membrane, is chosen from the group consisting of: alpha-adrenergic agonists, beta-adrenergic agonists, beta-adrenergic blockers, nerve agents for smoking cessation, anticholinergic agents, antiepileptic agents; anti-Parkinson agents, bronchodilators; narcotic antagonists, guanidine derivatives, quinazoline derivatives, reserpine derivatives, and sulfonamide derivatives.

In particular, an active pharmaceutical ingredient which is soluble in the solvent system, compatible with said solvent system, and suitable for delivery to the skin or a mucosal membrane, is chosen from the group consisting of: anti-inflammatory drugs, antiviral drugs, antibacterial drugs, antiparasitic drugs, anti-psoriatic drugs, drugs with effect on pain, drugs with effect on skin microcirculation, drugs with effect on formation on scars, drugs with effect on eczema, drugs with effect on perspiration, drugs with effect on growth of hair, drugs with effect on wound healing, drugs with effect on visible skin properties, drugs with effect on comedone closure, drugs with effect on skin barrier function, and drugs with effect on itching.

It is conceived that APIs can be chosen, which currently are identified for specific indications, but which when administered in a dermal, transdermal and/or mucosal formulation, are effective to alleviate, treat or prevent another indication, currently not associated with that API.

It is further conceived that APIs can be used in combination, and formulated together in the same dermal, transdermal and/or mucosal formulation, under the condition that both, in the case of two APIs, or all, in the case of three or more, are soluble in and compatible with the chosen solvent and/or solvent system.

According to an embodiment, the API is chosen from aciclovir, allopurinol, am itriptylinoxide, amlodipine, azathioprine, baclofen, bambuterol, beclometason dipropionate, benzoyl peroxide (BPO), betametason dipropionate, bibrocathole, budesonide, calcifediol, calcitriol, captopril, celecoxib, cilazepril (cilazapril), dapsone, dexametasone, doxepin, ergocalciferol, ethambutol, etodolac, felbinac, fenoprofen, flunisolide, fluticasone, ibuprofen, meloxicam, minoxidil, mitoxantrone, mometasone, moxisylyte, nabumetone, piracetam, piroxicam, propylhexedrine, pyritinol, salicylic acid, simvastatin, spironolactone, tenoxicam, terbinafine, terbutaline, tocopherols, urea, as well as compounds exhibiting comparable solubility as these, and combinations thereof.

According to an embodiment, based on available solubility data, the API is preferably chosen from the group consisting of: aciclovir, allopurinol, azathioprine, bambuterol, bibrocathol, budesonide, calcitriol, captopril, celecoxib, ethambutol, flunisolide, fluticasone, meloxicam, minoxidil, mitoxantrone, mometasone, piracetam, piroxicam, tenoxicam, terbinafine, terbutaline, salicylic acid, and spironolactone, including combinations thereof.

Most preferably the API is chosen from aciclovir, benzoyl peroxide, mometasone, piracetam, salicylic acid and spironolactone.

### The Anti-Solvent

The third component of the dermal, transdermal and/or mucosal formulation, the anti-solvent, is chosen from pharmaceutically acceptable solvents which are compatible with the API and the solvent and/or solvent system, but which are capable of precipitating the API, or in other word, forcing a significant part of the API into the solid state. Conversely, the API becomes dissolved in the solvent when the anti-solvent evaporates. Preferably the anti-solvent has a vapor pressure of more than 1 kPa at 25° C.

Preferably the anti-solvent is chosen from the group consisting of pentane, heptane, ethyl acetate, butyl acetate, propyl acetate, methyl acetate, acetone, methyl ethyl ketone, methyl isobutyl ketone, 3-methyl-2-butanone and water, and combinations thereof.

More preferably the anti-solvent comprises at least one compound selected from the group consisting of butyl acetate, ethyl acetate, water and combinations thereof.

Preferably the anti-solvent is an ester, most preferably an ester selected from the group consisting of methyl acetate, ethyl acetate, butyl acetate and propyl acetate.

The amount of anti-solvent is preferably from about 50% to 99%, more preferably from about 70% to 98% and most preferably from about 80% to 96% of the total composition.

### Additional Components

In addition, the formulation may include additional components or excipients well known to a person skilled in the art.

For example, so called permeation enhancers may be additionally included in the pharmaceutical formulation. They can be chosen from the group of enhancers suitable for use in a dermal, transdermal and/or mucosal formulation, provided that they are compatible with the API, the solvent and/or solvent system, as well as with the anti-solvent.

The pharmaceutical formulation of the invention may further include a gelling agent or thickener, in order to provide a suitable viscosity of the product during storage and in use. They can be chosen from the group of gelling agents or thickeners suitable for use in a dermal, transdermal and/or mucosal formulation, provided that they are compatible with the API, the solvent and/or solvent system, as well as with the anti-solvent.

Where necessary, for example in cases where the dermal, transdermal and/or mucosal formulation comprises water, it may be suitable to include a preservative. The preservative can be chosen from the group of preservatives suitable for use in a dermal, transdermal and/or mucosal formulation, provided that they are compatible with the API, the solvent and/or solvent system, as well as with the anti-solvent.

The dermal, transdermal and/or mucosal formulation may further comprise an antioxidant to further enhance the stability of the product. The antioxidant can be chosen from the group of antioxidants suitable for use in a dermal, transdermal and/or mucosal formulation, provided that they are compatible with the API, and soluble at least in the solvent and/or solvent system. Examples include, but are not limited to, tocopherol and derivatives thereof, ascorbic acid and derivatives thereof, butylated hydroxyanisole, butylated hydroxytoluene, fumaric acid, malic acid, propyl gallate, metabisulfates and derivatives thereof. The antioxidant is present from about 0.001% to about 5.0% depending on the type of compound.

Further, the formulation may comprise buffers such as carbonate buffers, citrate buffers, phosphate buffers, acetate buffers, hydrochloric acid, lactic acid, tartaric acid, diethylamine, triethylamine, diisopropylamine, aminomethylamine. However, other buffers as known in the art may be included, provided that they are compatible with the API, the solvent and/or solvent system, as well as with the anti-solvent.

A formulation according to this invention can be prepared with chelating agents exemplified by, but not limited to, EDTA or its derivatives and phosphonic acids.

When the dermal, transdermal and/or mucosal formulation is a spray, a propellant such as nitrous oxide, carbon dioxide or hydroflouralkanes (HFA) (HFA 134a (1,1,1,2,-tetrafluoroethane) or HFA 227 (1,1,1,2,3,3,3-heptafluoropropane)) may be included.

### PREFERRED EMBODIMENTS

According to a preferred embodiment, the active pharmaceutical ingredient is one which is soluble in an alcohol, and where the dermal, transdermal and/or mucosal formulation comprises an alcohol and where the anti-solvent preferably comprises an ester or water or mixtures thereof. Examples of active pharmaceutical ingredients which are soluble in alcohols include aciclovir, benzoyl peroxide, mometasone, piracetam, salicylic acid and spironolactone.

Preferably the alcohol is a pharmaceutically acceptable alcohol with a vapor pressure of less than 1 kPa at 25° C. More preferably the alcohol has a vapor pressure of less than 0.8 kPa at 25° C., even more preferably less than 0.5 kPa at 25° C. Such alcohols include but are not limited to di- or polyhydric alcohols like 1,2-propanediol (propylene glycol), butanediol, pentanediol, hexanediol, oleyl alcohol and glycerol.

Suitable components for inclusion in the anti-solvent are pharmaceutically acceptable components with a vapor pressure of more than 1 kPa at 25° C. Preferred components for inclusion in the anti-solvent are components selected from the group consisting of pentane, heptane, ethyl acetate, butyl acetate, propyl acetate, methyl acetate, acetone, methyl ethyl ketone, methyl isobutyl ketone, 3-methyl-2-butanone and water, or mixtures thereof. When the dermal, transdermal and/or mucosal formulation is a spray a propellant such as nitrous oxide, carbon dioxide or hydroflouralkanes (HFA) (HFA 134a (1,1,1,2,-tetrafluoroethane) or HFA 227 (1,1,1,2,3,3,3-heptafluoropropane)) may be included.

According to one embodiment, aciclovir is dissolved in a propanediol based solvent system, and precipitated with ethyl acetate as the anti-solvent. In this composition, aciclovir is present at about 0.2-2.0%, the solvent system at about 3-6%, and the anti-solvent at about 90-95% of the total composition.

According to another embodiment, benzoyl peroxide is dissolved in a polyethylene glycol based solvent system, and precipitated with a mixture of ethyl acetate and heptane as the anti-solvent. In this composition, benzoyl peroxide is present at about 0.1-1.0%, the solvent system at about 8-12%, and the anti-solvent at about 80-95% of the total composition.

According to yet another embodiment, mometasone is dissolved in a dipropylene glycol based solvent system, and precipitated with a mixture of ethanol and water as the anti-solvent. In this composition, mometasone is present at about 0.01-1.0%, the solvent system at about 4-10%, and the anti-solvent at about 80-95% of the total composition.

According to yet another embodiment, spironolactone is dissolved in a polyethylene glycol based solvent system, and precipitated with a mixture of ethanol and water as the anti-solvent. In this composition, spironolactone is present at about 0.1-2%, the solvent system at about 4-10%, and the anti-solvent at about 88-96% of the total composition.

According to yet another embodiment, piracetam is dissolved in a propylene glycol based solvent system, and precipitated with ethyl acetate as the anti-solvent. In this composition, piracetam is present at about 0.01-1.0%, the solvent system at about 4-12%, and the anti-solvent at about 87-95% of the total composition.

According to yet another embodiment, salicylic acid is dissolved in a propanediol based solvent system, and precipitated with ethyl acetate as the anti-solvent. In this composition, salicylic acid is present at about 5-15%, the solvent system at about 5-15%, and the anti-solvent at about 70-90% of the total composition.

### EXAMPLES

### Example 1

### Aciclovir was Dissolved in Propanediol and Precipitated with Ethyl Acetate

Aciclovir, an antiviral drug, was dissolved in a mixture of an acid and an alcohol according to Table 1.

TABLE 1
Composition comprising aciclovir

| | | |
|---|---|---|
| Aciclovir | 0.75 | g |
| Citric acid | 5 | g |
| 1,2-propanediol | 4.25 | g |
| Ethyl acetate | 90 | g |

First, citric acid, and then aciclovir, was dissolved in 1,2-propanediol while heating to 50° C. When ethyl acetate was added to the solution, a precipitation of aciclovir was observed. The product was stored as a suspension and when applied to a microscope glass slide, ethyl acetate evaporated and a transparent solution of aciclovir in citric acid and 1,2-propanediol remained. The presence of a clear, transparent solution shows that aciclovir had dissolved.

### Example 2

### Benzoyl Peroxide was Dissolved in Polyethylene Glycol and Precipitated with Ethyl Acetate and Heptane

Benzoyl peroxide was tested in a composition according to Table 2.

TABLE 2
Composition comprising benzoyl peroxide

| | | |
|---|---|---|
| Benzoyl peroxide | 0.25 | g |
| Polyethylene glycol 400 | 9.75 | g |
| Ethyl acetate | 45 | g |
| Heptane | 45 | g |

Benzoyl peroxide was dissolved in polyethylene glycol 400 and a anti-solvent mixture of ethyl acetate and heptane was added to form a suspension. The suspension was applied on a microscope glass slide and the anti-solvents allowed to evaporate. Benzoyl peroxide dissolved as the anti-solvent consisting of ethyl acetate and heptane evaporated.

### Example 3

### Mometasone was Dissolved in Dipropylene Glycol and Precipitated with Ethanol and Water

Mometasone was tested in a composition according to Table 3.

TABLE 3
Composition comprising mometasone

| | | |
|---|---|---|
| Mometasone furoate | 0.07 | g |
| Dipropylene glycol | 6.64 | g |
| Ethanol | 59.88 | g |
| Water | 33.42 | g |

Mometasone was first dissolved in the alcoholic solvent dipropylene glycol, whereupon a mixture of ethanol and water was prepared, before addition to the mometasone solution. A precipitation of mometasone was formed. When applying the suspension to a microscope glass slide the anti-solvent combination evaporated and a clear solution of mometasone in dipropylene glycol was formed.

### Example 4

### Spironolactone was Dissolved in Polyethylene Glycol and Precipitated with Ethyl Acetate and Heptane

In this experiment, spironolactone was dissolved in polyethylene glycol. In another vessel ethyl acetate and heptane were mixed, and when added to the spironolactone solution, a suspension of spironolactone was formed. For the total composition, see Table 4.

TABLE 4
Composition comprising spironolactone

| | | |
|---|---|---|
| Spironolactone | 0.17 | g |
| Polyethylene glycol 400 | 5.56 | g |
| Ethyl acetate | 47.14 | g |
| Heptane | 47.14 | g |

When the suspension was applied on a microscope glass plate, the anti-solvents, ethyl acetate and heptanes, evaporated leaving a clear solution of spironolactone in polyethylene glycol. The concentration of spironolactone in the remaining solution was about 3%.

### Example 5

### Piracetam can be Dissolved in Propylene Glycol and Precipitated with Ethyl Acetate

A mixture of propylene glycol with urea and lactic acid was made, and when a clear solution was generated, piracetam was added and dissolved while stirring. Ethyl acetate was charged and a suspension was formed. For the total composition, see Table 5. When the suspension was allowed to evaporate on a microscope glass slide, a solution of piracetam was formed.

TABLE 5
Composition comprising piracetam

| | | |
|---|---|---|
| Piracetam | 0.05 | g |
| Propylene glycol | 6.98 | g |
| Urea | 1.99 | g |
| Lactic acid | 1.00 | g |
| Ethyl acetate | 89.99 | g |

### Example 6

### Penetration Test of Spironolactone Composition

Spironolactone, an agent intended for topical treatment, was dissolved in an alcohol solvent and ethyl acetate was added to make a suspension. For the total composition, see Table 6a.

TABLE 6a
Composition comprising spironolactone

| | | |
|---|---|---|
| Spironolactone | 0.3 | g |
| Polyethylene glycol 400 | 9.7 | g |
| Ethyl acetate | 45 | g |
| Heptane | 45 | g |

The functionality of the formulation was tested by evaporating the anti-solvent ethyl acetate. The presence of a clear solution after evaporation of ethanol indicated that spironolactone had dissolved. This formulation was used for penetration testing. When applied to the skin, spironolactone is intended to penetrate through the statum corneum into epidermis/dermis and not through the skin.

Thus, a penetration study was performed using the Franz cell set-up. Such equipment is commercially available, for example from PermeGear Inc. (Hellertown, Pa., USA). In this study, a model V9-CB, compact 9-station stirrer with black anodized aluminum cell holders for jacketed Franz Cells was used. The glass cells are 9 mm jacketed Franz cells with flat ground joints, clear glass, ml receptor volume, stir bar and cell top.

In this set-up, dermis was removed from full thickness pig skin and the remaining membrane consisting of stratum corneum and epidermis was mounted in the cell. The receptor fluid was phosphate buffer at pH 7.4 and 32° C. An amount of the experimental formulation corresponding to 50 mg of product and 1.5 mg of active substance after evaporation was applied. Samples were withdrawn at 2, 4, 6 and 24 hours, and after 6 hours it was found that 0.5 mg of spironolactone had passed the skin membrane (33% of the dose). The experiment was performed at 34° C. and analysis of spironolactone was performed by HPLC. The results are presented in Table 6b below:

TABLE 6b
Cumulative penetration of spironolactone through
stratum corneum/epidermis in μg/cm²

| | Formulation/Time (h) | | | | |
|---|---|---|---|---|---|
| | 0 | 2 | 4 | 6 | 24 |
| Spironolactone | 0 | 281 | 381 | 440 | 466 |
| (μg/cm²) | | | | | |

### Example 7

### Comparative Example Using Two Salicylic Acid Containing Formulations

A salicylic acid containing formulation according to an embodiment of the invention was prepared, and compared to a commercial wart remover (Stark DnE® from Den Norske Eterfabrikk A/S, Oslo, Norway). The compositions of both formulations are disclosed in Table 7 below:

TABLE 7
Composition of two salicylic acid containing formulations

| | Experimental | | Commercial | |
|---|---|---|---|---|
| | Formulation A | | wart remover | |
| | Batch No. | | product B | |
| | ISM11192 | | Stark DnE ® | |
| | Wet | | Wet | |
| Substance | % | Evaporated | % | Evaporated |
| Salicylic acid | 5 | 23.8 | 20 | 43.5 |
| Urea | 2 | 9.5 | 0 | |
| Lactic acid | 1 | 4.8 | 20 | 43.5 |
| 1,2-propanediol | 7 | 33.3 | 0 | |
| Nitrocellulose | 6 | 28.5 | 6 | 13 |
| Ethyl acetate | To 100% | | — | |
| Diethyl ether | — | | To 100% | |

The experimental formulation was manufactured by dissolution of the active, salicylic acid in a solution of propane-1,2-diol, lactic acid and urea. Nitrocellulose was dissolved in ethyl acetate and the two clear solutions were mixed to create a suspension of salicylic acid. When exposing a thin layer of the formulation to ambient air, the anti-solvent ethyl acetate evaporated and left a clear film of nitrocellulose, 1,2-propanediol, urea, lactic acid and salicylic acid, all in solution.

A penetration study was performed using the Franz cells describe above. Hoof membranes were collected from bovine hoof from an abattoir (SLP, Helsingborg, Sweden). Only membranes from the sole of the bovine hoof were been used. The thickness of the membranes was 100 μm and they were prepared by gluing 3×3 cm cubes of the hoof sole to a metacrylate block and making slices of 100 μm thickness with a dermatome. The hoof membranes were frozen and stored at −80° C. until use.

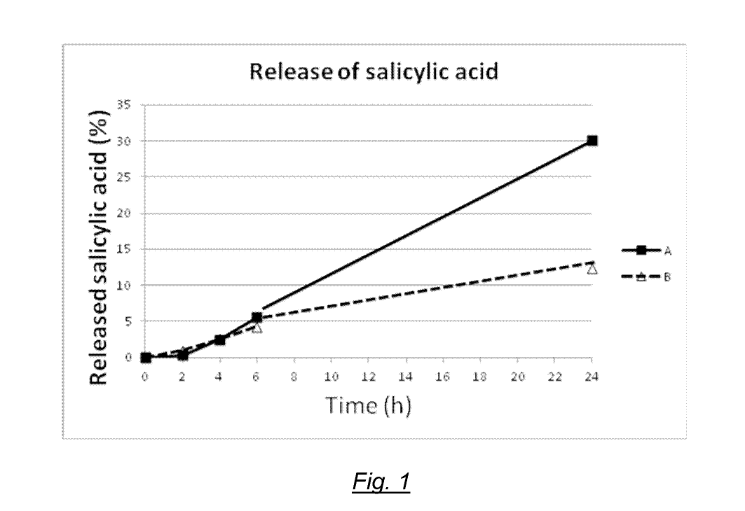

The degree of penetration was expressed as percentage of the salicylic acid delivered through the membrane. The penetration of salicylic acid from formulation A, and embodiment of the invention, through hoof material, intending to mimic the keratinized tissue of warts, revealed fast delivery of salicylic acid through the hoof keratin membrane. The fraction of the dose delivered through the membrane after 24 hours was about 30% for formulation A, and only about 12% for formulation B. See FIG. 1.

### Example 8

### Comparative Example Using Two Different Piracetam-Containing Formulations

Two piracetam-containing formulations were prepared, having the ingredients shown in Table 8.

TABLE 8
Two piracetam containing formulations

| | C | | D | |
|---|---|---|---|---|
| Ingredients | (Batch No. ISM12006) | | (Batch No. ISM12007) | |
| Propylene glycol | 7 | g | 10 | g |
| Urea | 2 | g | 0 | |
| Lactic acid | 1 | g | 0 | |
| Piracetam | 0.5 | g | 0.5 | g |
| Ethyl acetate | 89.5 | g | 89.5 | g |

The formulations were manufactured by dissolution in the respective solvents, propylene glycol or a combination of propylene glycol, urea and lactic acid, followed by the addition of ethyl acetate to create a suspension. The penetration of piracetam through full thickness pig skin was determined using the Bronaugh flow-through diffusion cell system (equipment from PermeGear Inc., Hellertown, Pa., USA) for skin penetration studies.

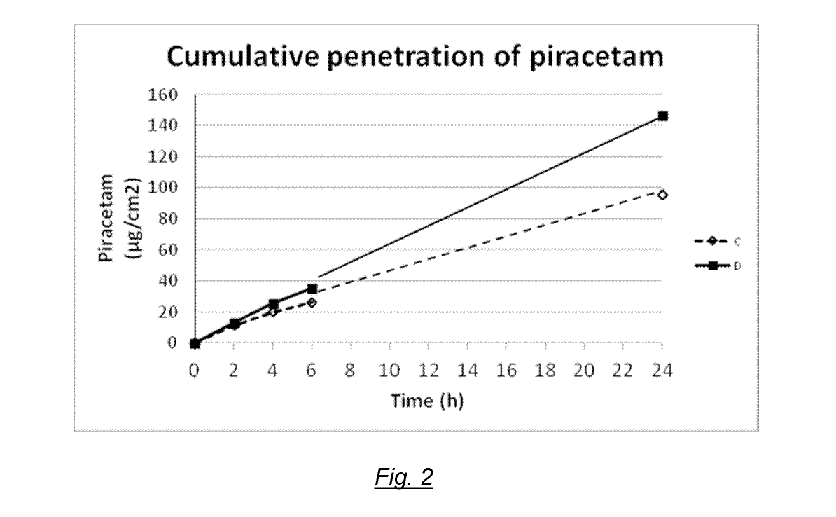

The results of the penetration study are presented in FIG. 2 below.

Although the invention has been described with regard to its preferred embodiments, which constitute the best mode presently known to the inventors, it should be understood that various changes and modifications as would be obvious to one having the ordinary skill in this art may be made without departing from the scope of the invention as set forth in the claims appended hereto.

1. A dermal, transdermal, and/or mucosal formulation comprising:
at least one active pharmaceutical ingredient selected from aciclovir, benzoyl peroxide, mometasone, piracetam, salicylic acid and spironolactone;
a pharmaceutically acceptable solvent and/or solvent system; and
a pharmaceutically acceptable anti-solvent, characterized in that
(a) the active pharmaceutical ingredient is substantially in the solid state in said formulation in the presence of said anti-solvent;
(b) the active pharmaceutical ingredient is soluble in the solvent and/or solvent system in the absence of said anti-solvent,
(c) the solvent and/or solvent system includes a dihydric or polyhydric alcohol, and
(d) the anti-solvent comprises an ester or water or mixtures thereof.

2. (canceled)

3. (canceled)

4. The formulation according to claim 1, wherein the pharmaceutically acceptable solvent and/or solvent system includes an unsaturated aliphatic dihydric or trihydric alcohol.

5. The formulation according to claim 1, wherein the pharmaceutically acceptable solvent and/or solvent system includes an unsaturated dihydric or trihydric alicyclic alcohol.

6. The formulation according to claim 1, wherein the pharmaceutically acceptable solvent and/or solvent system includes an alcohol chosen from 1,2-propanediol, butanediol, pentanediol, hexanediol, polyethylene glycol, glycerol and mixtures thereof.

7. The formulation according to claim 1, wherein the formulation further comprises a solvent chosen from inositol, xylitol, sorbitol and mannitol.

8. (canceled)

9. The formulation according to claim 1, wherein the pharmaceutically acceptable solvent and/or solvent system has a vapor pressure of less than 1 kPa at 25° C.

10. The formulation according to claim 9, wherein the pharmaceutically acceptable solvent and/or solvent system has a vapor pressure of less than 0.8 kPa at 25° C.

11. The formulation according to claim 10, wherein the pharmaceutically acceptable solvent and/or solvent system has a vapor pressure of less than 0.5 kPa at 25° C.

12. The formulation according to claim 1, wherein the anti-solvent is chosen from pharmaceutically acceptable compounds having vapor pressure of more than 1 kPa at 25° C.

13. The formulation according to claim 12, wherein the anti-solvent comprises at least one compound selected from the group consisting of, ethyl acetate, butyl acetate, propyl acetate, methyl acetate, water and combinations thereof.

14. The formulation according to claim 13, wherein the anti-solvent comprises at least one compound selected from the group consisting of butyl acetate, ethyl acetate, water and combinations thereof.

15. (canceled)

16. The formulation according to claim 13, wherein the anti-solvent comprises methyl acetate, ethyl acetate, butyl acetate or propyl acetate.

17. The formulation according to claim 1, wherein the formulation is in the form of a cream, ointment, paste, lotion, gel, foam or spray.

18. A method for increasing the stability of an active pharmaceutical ingredient selected from aciclovir, benzoyl peroxide, mometasone, piracetam, salicylic acid and spironolactone in a dermal and/or mucosal formulation, characterized in that the active pharmaceutical ingredient is dissolved in a solvent and/or solvent system, whereupon an anti-solvent is added, said anti-solvent being effective to substantially precipitate said active pharmaceutical ingredient, and wherein said solvent and/or solvent system comprises a dihydric or polyhydric alcohol, and the anti-solvent comprises an ester or water or mixtures thereof.

19. (canceled)

20. (canceled)

21. The method according to claim 18, wherein the pharmaceutically acceptable solvent and/or solvent system includes an unsaturated aliphatic dihydric or trihydric alcohol.

22. The method according to claim 18, wherein the pharmaceutically acceptable solvent and/or solvent system includes an unsaturated dihydric or trihydric alicyclic alcohol.

23. The method according to claim 18, wherein the pharmaceutically acceptable solvent and/or solvent system includes an alcohol chosen from 1,2-propanediol, butanediol, pentanediol, hexanediol, polyethylene glycol, glycerol and mixtures thereof.

24. The method according to claim 18, wherein the formulation further comprises a solvent chosen from inositol, xylitol, sorbitol and mannitol.

25. (canceled)

26. The method according to claim 18, wherein the pharmaceutically acceptable solvent and/or solvent system has a vapor pressure of less than 1 kPa at 25° C.

27. The method according to claim 26, wherein the pharmaceutically acceptable solvent and/or solvent system has a vapor pressure of less than 0.8 kPa at 25° C.

28. The method according to claim 27, wherein the pharmaceutically acceptable solvent and/or solvent system has a vapor pressure of less than 0.5 kPa at 25° C.

29. The method according to claim 18, wherein the anti-solvent is chosen from pharmaceutically acceptable compounds having vapor pressure of more than 1 kPa at 25° C.

30. The method according to claim 18, wherein the anti-solvent comprises at least one compound selected from the group consisting of ethyl acetate, butyl acetate, propyl acetate, methyl acetate, water, and combinations thereof.

31. The method according to claim 30, wherein the anti-solvent comprises at least one compound selected from the group consisting of butyl acetate, ethyl acetate, water and combinations thereof.

32. (canceled)

33. The method according to claim 30 wherein the anti-solvent comprises methyl acetate, ethyl acetate, butyl acetate and or propyl acetate.

34. The method according to claim 18, wherein the formulation is in the form of a cream, ointment, paste, lotion, gel, foam or spray.

35. The formulation according to claim 10, wherein the anti-solvent comprises water.

36. The formulation according to claim 31, wherein the anti-solvent comprises water.
